(19) United States
(12) Patent Application Publication
(10) Pub. No.: US 2006/0111748 A1
(43) Pub. Date: May 25, 2006

(54) Defibrillator

(75) Inventor: Jurgen Bucher, Rottweil (DE)

Correspondence Address: Pauley Petersen & Erickson, Suite 365, 2800 W Higgins Road, Hoffman Estates, IL, 60195, US

(73) Assignee: Metrax GmbH, DE-78628 Rottweil (DE)

(21) Appl. No.: 10/540,233

(22) PCT Filed: Nov. 4, 2003
(86) PCT No.: PCT/EP03/12264

§ 371 Date: Jun. 21, 2005

(30) Foreign Application Priority Data
Nov. 5, 2002 (DE) 102 51 344.9

(51) Int. Cl. A61N 1/39

(52) U.S. Cl. 607/5

(57) ABSTRACT

A defibrillator having an electronic system arranged in a housing and electrodes which can be connected to the system and are to be placed on the patient. This invention provides a user-friendly defibrillator. Thus, the electrodes are received in a chamber provided on the inner side of a cover which can be opened or removed.

### BACKGROUND OF THE INVENTION

1. Field of the Invention

This invention relates to a defibrillator with an electronic device arranged in a housing and having electrodes which can be connected thereto and can be applied to a patient.

2. Discussion of Related Art

A defibrillator is taught by European Patent Reference EP 0 801 959 A2. There, patient electrodes are connected by a connecting cable to a socket arranged on an outside of a housing wall. Furthermore, there is a control panel with operating and display elements on an outside of the housing. Such defibrillators are more and more often positioned as emergency devices in buildings accessible to the public or other suitable locations, so that they are quickly available in case of an emergency. Such defibrillators should be designed as simply as possible, so that they can also be operated by an untrained user.

Patient electrodes packed in a vacuum-tight manner are taught by U.S. Pat. No. 5,502,894.

### SUMMARY OF THE INVENTION

One object of this invention is to provide a defibrillator of the type mentioned above but which is particularly user-friendly.

This object is achieved with a defibrillator having characteristics discussed in this specification and in the claims. Thus, the electrodes are received in a chamber formed on the inside of a cover which can be flipped open or removed.

The electrodes to be applied to a patient are not only protectively housed in the device by these measures, but are also immediately available to a user in the event of an emergency after the user has simply removed the cover from the housing.

The simple operation and dependable functioning are assisted if in the unused state of the defibrillator the electrodes are connected by a connecting cable and a plug connected therewith to a connection socket of the housing connected with the electronic device in a hollow chamber covered in an unused state by the cover.

In order to assure dependable functioning over a prolonged period of time the measures are advantageous, wherein the electrodes are received by a vacuum-sealed electrode receptacle in the chamber of the cover. It is also possible to simultaneously program cyclically occurring electrode test processes, wherein the electrodes are suitably designed and arranged and are provided in a known manner with a gel layer on their active surface. Together with their pickup, the electrodes can be easily taken from the chamber in the cover, or are already released from the housing when taking the cover off, because the electrode receptacle is torn open because of its structure and arrangement in the housing.

For the operation a handle is arranged on the outside of the cover, which can be grasped by a user and by which the cover can be torn off the housing.

In one advantageous embodiment, the handle is a pull strap connected with the cover or the electrode receptacle. If, for example, the pull strap is fastened on the electrode receptacle, it is replaced after a use of the defibrillator when the chamber is equipped with a fresh electrode receptacle, wherein the pull strap is conducted from the inside of the cover to the outside.

Embodiments advantageous for operation include the cover having holding elements which, for fixing it on the housing, are latched, clipped or snapped into counter-holding elements arranged on the outside of the housing. In this case, the holding elements and/or the counter-holding elements can be advantageously formed on the cover or the housing.

With further chambers formed and further removable operating utensils received on an inside of the cover, there is a user-friendly design. A razor, gloves, a brush, or the like are operating utensils which are useful when the defibrillator is used.

It can be advantageous for an ordered housing and definite closure on the inside of the cover for the at least one chamber with the electrodes and possibly further operating utensils to be covered by a removable inner cover element.

The steps are advantageous for functioning and simple operation, wherein a housing wall at the front, which in the unused state is covered by the cover and in the used state is released, is embodied as a control panel with at least one triggering element for defibrillation and user guide elements.

It is helpful for simple use that information for the actuation of the handle is arranged on the outside of the cover. For example, the information can be an easily understandable symbol on the outside of the cover.

### BRIEF DESCRIPTION OF THE DRAWINGS

This invention is explained in greater detail in view of an exemplary embodiment and by making reference to the drawings, wherein:

FIG. 1 shows a defibrillator with a holder in a perspective plan view;

FIG. 2 shows a cover of the defibrillator in FIG. 1, in a perspective plan view;

FIG. 3 shows the defibrillator in FIG. 1, with the cover removed;

FIGS. 4a, 4b and 4c show the inside of the cover shown in FIG. 2, in three different states of being equipped;

FIG. 5 shows a partial sectional view of the defibrillator in the area of or near a holding section of the cover;

FIG. 6 shows a perspective view of the defibrillator with the cover removed; and

FIGS. 7a to 7f show different views of the cover.

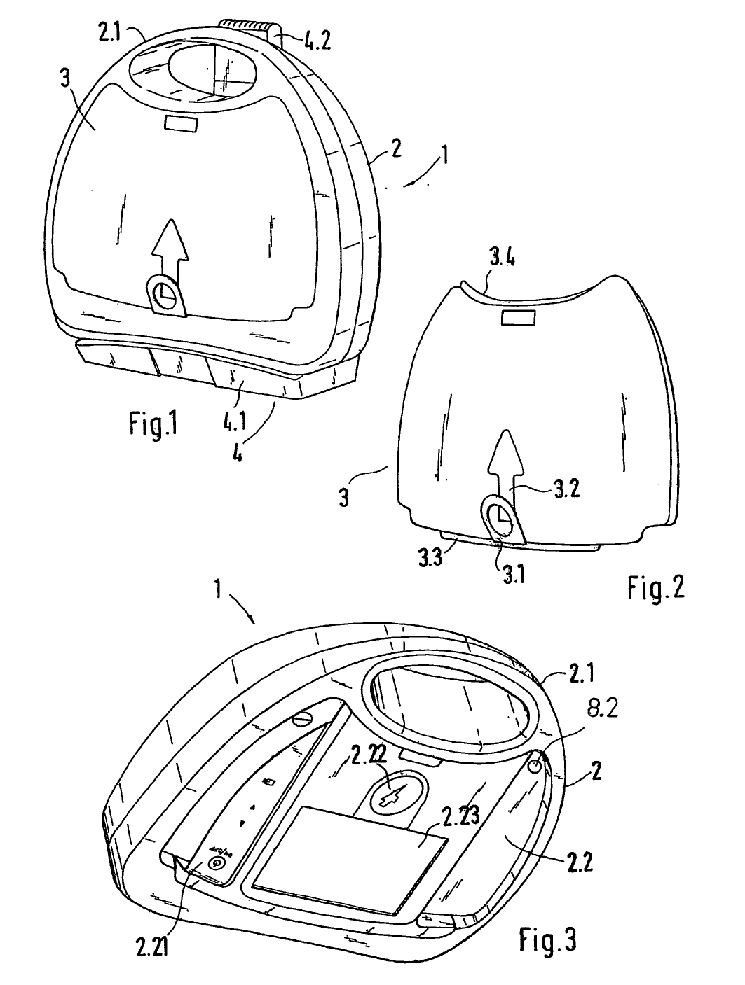

### DESCRIPTION OF PREFERRED EMBODIMENTS

A defibrillator 1 with a housing 2, into whose front a cover 3 is inserted and which has a holder 4 with a base element 4.1 and rear support element or holding element 4.2, is shown in FIG. 1. Such a defibrillator, a so-called automatic external defibrillator (AED for short) can be operated by an untrained user in case of an emergency and is preferably positioned in an easily accessible location.

The housing 2 with outer contours designed to be rounded, has an integrated handle element 2.1 in an upper area, and on a front has a control panel which, for example, has a control section 2.21, a trigger element 2.22 for defibrillation and a display unit 2.23, and is arranged in a depression recessed in respect to its border. The cover 3 is fitted into the border in order to form with its front a homogeneous termination, slightly convexly arched toward the exterior. The depression, and thus in the unused state also the cover 3, are surrounded in a border-like manner on all sides by the remaining area of the front of the housing. The base element 4.1 of the holder 4 is attached to the underside of the border.

In the transition area between the lower border section or the upper or a lateral border section of the housing 2 and the cover 3, a grasping means in the form of a pull strap 3.1 is arranged, by which the inserted cover 3 can be rapidly and simply removed from the housing 2 by obliquely pulling upward, or possibly downward or laterally toward the front, as indicated by the information means in the form of an information arrow 3.2 applied to the front of the cover 3.

The cover 3 is fixed in place on the housing 2 by one or several holding sections 3.3 formed in the upper edge area, and one or more further holding sections 3.4 formed on the lower edge of the cover 3, in matched complementary counter-holding elements in the transition area of the respective border sections to the depression. Other holding sections are also conceivable, for example formed on the rear of the cover 3, and correspondingly arranged counter-holding sections in the bottom of the depression. Because of its elastic force, the plate-like cover 3 made of plastic and slightly convexly arched forward is dependably held on the housing 2 and can also be easily removed therefrom in case of emergency. Magnetic holding sections and counter-holding elements, or those acting in accordance with the principle of a burr-type closure, are also possible.

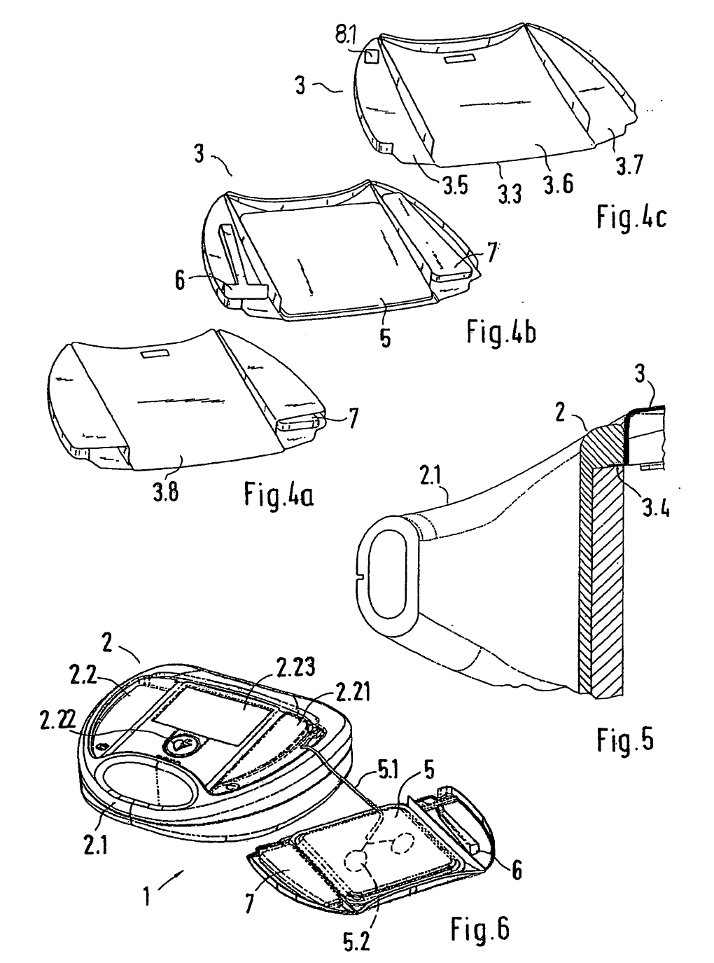

Furthermore, a sensor device is advantageously provided, by which the defibrillator 1 is switched on as soon as the lid-like cover 3 is removed from the housing 2. For example, the sensor device has a magnetically or opto-electrically or electro-mechanically functioning switching device, wherein a sensor element 8.1, for example a permanent magnet or optically acting element or mechanical triggering element, arranged on the cover 3 acts together with a sensor element 8.2, for example a magnetic field sensor element, a photo-electrically reacting sensor element or a mechanically activable switch or key, arranged on a corresponding housing location, as shown in FIGS. 3 and 4C. Thus, a separate on/off switch can be omitted and the device is ready for triggering the defibrillation pulse after the electrodes are correctly placed.

Several chambers are formed on the back of the cover 3, which are separated from each other by formed-on strips, as shown in FIG. 4c. Electrodes 5.2, such as shown in FIG. 6, are placed into a central chamber 3.6, which themselves have preferably been placed into a vacuum-sealed, or at least dust-sealed or moisture-sealed electrode receptacle 5 or packaging, wherein a connecting cable 5.1, which is connected with the electrodes 5.2 extends sealed out of the electrode receptacle 5 and is connected with its other end, to which a plug 5.3 is attached, in a socket in the edge of the depression. The socket is electrically connected with an electronic device of the defibrillator 1 arranged in the interior of the housing 2.

Other chambers 3.5 and 3.7 are arranged at the sides of the central chamber 3.6, in which suitable operating utensils for the use of the defibrillator 1 are located, for example a razor 6, gloves and a brush 7, as shown in FIG. 4b. The chambers 3.5, 3.6 and 3.7, along with the utensils stored therein, are covered on the inside by a cover element 3.8. With a window arranged in the upper portion of the cover 3 it is possible to make a visual check from the outside to determine whether the chamber 3.6 is re-equipped with a fresh electrode package 5, for example after the defibrillator 1 is used. The pull strap 3.1 can be arranged, for example, on the cover 3 or on the electrode receptacle 5, wherein it is passed through the lower, or possibly upper or lateral edge of the cover 3 and the lower border section of the housing 2. If attached to the electrode receptacle 5, it is also possible to check whether one exists on the inside of the cover 3. Also, when pulling the pull strap 3.1, the user already has the electrode package 5 with the electrodes in his hand, so that a rapid operation is made easier.

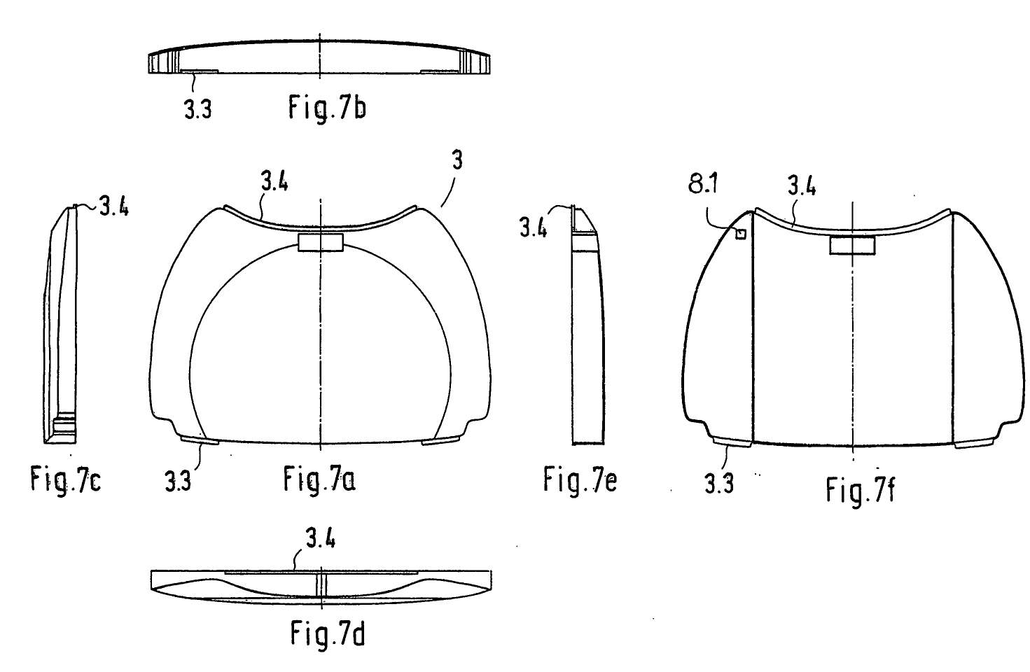

The enlarged portion in FIG. 5 shows the holding section 3.4 of the cover 3 placed on the border of the housing 2. The six plan views of the partial illustrations of FIGS. 7a to 7f show the cover 3 in a front view, view from below, lateral view from the right, from below, lateral view from the left, as well as from a plan view from the rear, wherein the holding sections 3.4 at the lower edge and the further holding sections 3.4 at the upper edge are clearly shown.

1. A defibrillator having an electronic device arranged in a housing and electrodes connectible thereto and that can be applied to a patient,
the defibrillator comprising:
the electrodes (5.2) received in a chamber (3.6) formed on an inside of a cover (3) which can be one of flipped open and removed.

2. The defibrillator in accordance with claim 1, wherein in an unused state of the defibrillator (1) the electrodes (5.2) are connected by a connecting cable (5.1) and a plug (5.3) connected therewith to a connection socket of the housing (2) connected with the electronic device in a hollow chamber covered in the unused state by the cover.

3. The defibrillator in accordance with claim 2, wherein the electrodes (5.2) are received by one of a vacuum-sealed, a moisture-proof and a dust-proof electrode receptacle (5) in the chamber (3.6) of the cover (3).

4. The defibrillator in accordance with claim 3, wherein a handle (3.1) is arranged on an outside of the cover (3), which can be grasped by a user and by which the cover (3) can be torn off the housing (2).

5. The defibrillator in accordance with claim 4, wherein the handle is a pull strap (3.1) connected with one of the cover (3) and the electrode receptacle (5).

6. The defibrillator in accordance with claim 5, wherein the cover (3) has holding elements (3.3, 3.4) which for fixing on the housing (2) are one of latched, clipped and snapped into counter-holding elements arranged on the outside of the housing, or are magnetically held on the outside of the housing.

7. The defibrillator in accordance with claim 6, wherein further chambers are formed on the inside of the cover (3) and removable operating utensils (6, 7) are received.

8. The defibrillator in accordance with claim 7, wherein the at least one chamber (3.5, 3.6, 3.7) with at least one of the electrodes (5.2) and the further operating utensils (6, 7) is covered by a removable inner cover element (3.8).

9. The defibrillator in accordance with claim 8, wherein a housing wall at the front, which in the unused state is covered by the cover (3) and in the used state is released, is a control panel (2.2) with at least one triggering element (2.22) for defibrillation and user guide elements (2.22, 2.21).

10. The defibrillator in accordance with claim 9, wherein information (3.2) for actuation of the handle (3.1) is arranged on the outside of the cover (3).

11. The defibrillator in accordance with claim 10, wherein a sensor arrangement (8.1, 8.2) responds to the removal of the cover (3) for switching on the defibrillator (1).

12. The defibrillator in accordance with claim 1, wherein the electrodes (5.2) are received by one of a vacuum-sealed, a moisture-proof and a dust-proof electrode receptacle (5) in the chamber (3.6) of the cover (3).

13. The defibrillator in accordance with claim 1, wherein a handle (3.1) is arranged on an outside of the cover (3), which can be grasped by a user and by which the cover (3) can be torn off the housing (2).

14. The defibrillator in accordance with claim 13, wherein the handle is a pull strap (3.1) connected with one of the cover (3) and an electrode receptacle (5) of the cover (3).

15. The defibrillator in accordance with claim 1, wherein the cover (3) has holding elements (3.3, 3.4) which for fixing on the housing (2) are one of latched, clipped and snapped into counter-holding elements arranged on an outside of the housing, or are magnetically held on the outside of the housing.

16. The defibrillator in accordance with claim 1, wherein further chambers are formed on the inside of the cover (3) and removable operating utensils (6, 7) are received.

17. The defibrillator in accordance with claim 1, wherein the chamber (3.5, 3.6, 3.7) with at least one of the electrodes (5.2) and the further operating utensils (6, 7) is covered by a removable inner cover element (3.8).

18. The defibrillator in accordance with claim 1, wherein a housing wall at a front, which in an unused state is covered by the cover (3) and in a used state is released, is a control panel (2.2) with at least one triggering element (2.22) for defibrillation and user guide elements (2.22, 2.21).

19. The defibrillator in accordance with claim 4, wherein information (3.2) for actuation of the handle (3.1) is arranged on the outside of the cover (3).

20. The defibrillator in accordance with claim 1, wherein a sensor arrangement (8.1, 8.2) responds to a removal of the cover (3) for switching on the defibrillator (1).
